(19) United States
(12) Patent Application Publication
(10) Pub. No.: US 2014/0336612 A1
(43) Pub. Date: Nov. 13, 2014

(54) INFLATABLE SYSTEM FOR ENTERAL FEEDING DEVICE

(71) Applicant: Boston Scientific Scimed, Inc., Maple Grove, MN (US)

(72) Inventors: Shoshana FRYDLEWSKI, Framingham, MA (US); Ralph Barry, JR., Hudson, MA (US)

(73) Assignee: Boston Scientific Scimed, Inc., Maple Grove, MN (US)

(21) Appl. No.: 14/269,394

(22) Filed: May 5, 2014

Related U.S. Application Data
(60) Provisional application No. 61/821,268, filed on May 9, 2013.

(51) Int. Cl. A61L 31/14; A61M 25/10

(52) U.S. Cl. CPC A61L 31/14; A61M 25/1025; A61M 2205/3613
USPC 604/500; 604/102.02

(57) ABSTRACT

Systems and methods of providing an enteral feeding device within a human body, e.g., the stomach, via a stoma are disclosed. One or more systems may comprise a tube having distal and proximal ends, and a lumen extending therebetween, the lumen in communication with an inflatable member and an inflation port, and an inflation medium to supply the inflatable member. The inflation medium may comprise a fluid including a thermosensitive polymer.

### CROSS-REFERENCE TO RELATED APPLICATIONS

This application claims the benefit of priority from U.S. Provisional Application No. 61/821,268, filed on May 9, 2013, the entirety of which is incorporated by reference herein.

### TECHNICAL FIELD

The present disclosure generally relates to devices, systems, and methods useful in enteral feeding. More particularly, the present disclosure includes devices, systems, and methods of retaining gastrostomy inflatable devices inside a human body, such as within the stomach.

### BACKGROUND

Enteral feeding devices (or enteral medicating devices) assist a patient struggling with temporary and/or long-term medical conditions, such as cystic fibrosis, malabsorption, and gastric decompression. Under these conditions, the patient is unable to meet his/her nutritional needs by conventional eating, i.e., through the mouth, and instead the patient depends on an energy/medication supplement directly into the stomach or intestines. In such cases, an enteral feeding device or gastrointestinal device may be passed through the esophagus, or may be introduced directly into the stomach or intestinal space via a stoma or artificial opening through the abdominal wall.

These gastrointestinal devices may include an inflatable member to position the device within the stomach. The inflatable member is typically provided in the form of a balloon to firmly bolster and retain the device in the stomach and intestinal space for a period of time. The inflatable member thus keeps the device in place and prevents accidental removal. The patient therefore can enjoy an active lifestyle.

Balloons are typically formed of “soft” or elastomeric medical grade silicone, which is typically deflated for insertion through the stoma, and then inflated using sterile water or saline to keep the enteral feeding assembly in position. The walls of the silicone balloon may be porous such that the inflation fluid, e.g., water or saline, may leech through the walls, thereby reducing the volume of fluid in the balloon and increasing the likelihood of the balloon being dislocated or unintentionally removed from the stomach. Movement of the balloon from its proper position may also cause discomfort to the patient.

Additionally, the fluid volume of the balloon bolsters in conventional devices may require frequent inspection and monitoring to determine whether a sufficiently significant volume of water has escaped from the balloon that might lead to unintentional removal of the balloon from the stomach. Such continual monitoring is burdensome to the patient and healthcare providers who may need to check the balloon volume frequently to ensure sufficient inflation and proper positioning of the device.

### SUMMARY

Thus, there is a need for alternative systems to reduce the need to monitor the placement of gastrostomy devices over time, and to minimize or prevent unintentional removal of such devices. The present disclosure may address some or all of these benefits. For example, some embodiments inflate the balloon using a fluid including a water-soluble polymer and/or thermosensitive polymer or polymer mixture that increases in viscosity upon being heated, e.g., by human body heat. In some embodiments, the fluid is sufficiently viscous to enable inflation and deflation of the balloon in a time consistent with enteral feeding. In some embodiments, the fluid has molecular properties that enable it to avoid clogging the apparatus that supplies the fluid to the balloon, including but not limited to valves. In some other embodiments, the fluid has molecular properties preventing, or at least reducing the amount of, fluid leeching or evaporating from the balloon over time.

Embodiments of the present disclosure include an enteral feeding system comprising a tube having a distal end, a proximal end, and at least one lumen extending therebetween, the tube configured for positioning within a stoma of a human body; an inflation medium configured to increase in viscosity with increasing temperature; an inflatable member located adjacent to the distal end of the tube, the inflatable member being configured to pass through the stoma in a deflated state, and to not pass through the stoma in an inflated state; and an inflation port located proximate to the proximal end of the tube, the inflation port being in fluid communication with the inflatable member via the at least one lumen to enable supply of the inflation medium into the inflatable member, wherein the inflatable member is configured to allow human body heat to warm the inflation medium to be provided therein to increase the viscosity of the inflation medium.

Embodiments of the present disclosure may include one or more of the following features: the inflation medium may comprise a liquid capable of transitioning into a gel upon being heated; the inflation medium may comprise at least one polymer; the at least one polymer may be naturally-derived or synthetic; the inflation medium may comprise at least one homopolymer, copolymer, block copolymer, cross-linked polymer, or hydrophilic polymer; the inflation medium may comprise at least one of acrylic acid, maleic anhydride polymers, allylamine, ethyleneimine, oxazoline, polyvinyl alcohol, polyethylene glycol, dextran, or polyvinylpyrrolidone; or the inflation medium may comprise at least one thermosensitive polymer.

Embodiments of the present disclosure further include an enteral feeding system comprising a gastric tube having a distal end, a proximal end, and tube walls defining an inflation lumen and a feeding lumen, the feeding lumen opening at the distal end of the tube, wherein the gastric tube includes: an inflatable member located adjacent to the distal end of the tube; and an inflation port and a feeding port, each located adjacent to the proximal end of the tube, wherein the inflation port is in fluid communication with the inflatable member through the inflation lumen, and wherein the feeding port is in fluid communication with the feeding lumen; a syringe configured to house a volume of a fluid, wherein the fluid comprises at least one thermosensitive polymer configured to increase in viscosity with increasing temperature; and a feeding tube configured to attach in fluid communication with the feeding port, the feeding tube being capable of extending into a stomach through the feeding lumen.

Embodiments of the present disclosure may include one or more of the following features: the fluid may be configured to increase in viscosity into a gel or a solid upon being heated; the fluid may be water soluble; the fluid may comprise at least one homopolymer, copolymer, block copolymer, cross-linked polymer, or hydrophilic polymer; the at least one thermosensitive polymer may be naturally-derived or synthetic; the fluid may comprise at least one of acrylic acid, maleic anhydride polymers, allylamine, ethyleneimine, oxazoline, polyvinyl alcohol, polyethylene glycol, dextran, or polyvinylpyrrolidone; the fluid may comprise at least one poloxamer; or the kit may comprise at least one container including the fluid.

Embodiments of the present disclosure further include a method of supplying a material to a human body via a stoma. The method may include passing a medical device through the stoma, the medical device comprising: a tube having a distal end, a proximal end, and first and second lumens extending therebetween; a fluid including at least one thermosensitive polymer configured to increase in viscosity with increasing temperature; an inflatable member located adjacent to the distal end of the tube, the inflatable member being configured to pass through the stoma in a deflated state, and to not pass through the stoma in an inflated state; and an inflation port located proximate to the proximal end of the tube, the inflation port being in fluid communication with the inflatable member via the first lumen; injecting the fluid into the inflatable member to expand the inflatable member, wherein human body heat causes the fluid to increase in viscosity to secure the medical device at the stoma; and feeding the fluent materials to the stomach through the second lumen.

Embodiments of the present disclosure may include one or more of the following features: the method may comprise injecting water into the inflatable member to dissolve the fluid in the water; and deflating the inflatable member by drawing the water and fluid out of the inflatable member through the first lumen; the fluid may solidify into a gel upon being heated; the fluid may comprise at least one homopolymer, copolymer, block copolymer, cross-linked polymer, or hydrophilic polymer; or the fluid may comprise at least one poloxamer.

Embodiments are disclosed herein in the context of preventing a balloon from exiting a human stomach through the stomach's stoma by increasing the size/volume of the balloon (subsequent to its insertion) so that it no longer fits through the stoma. However, embodiments are not limited to this context, and are intended to include or otherwise cover other applications. For example, some embodiments can be directed to retaining a balloon in other parts of the human body that have nothing to do with enteral feeding.

### BRIEF DESCRIPTION OF THE DRAWINGS

The drawings illustrate the design and utility of exemplary embodiments of the present disclosure, in which similar elements are referred to by common reference numerals. In order to better appreciate how the above-disclosed and other advantages and objects of the present disclosure are obtained, a more particular description of the present inventions briefly described above will be rendered by reference to specific embodiments thereof, which are illustrated in the accompanying drawings.

FIG. 1 is a perspective view of an enteral feeding device including an exemplary inflation system, in accordance with the present disclosure.

FIG. 2A is a perspective view of a balloon in a deflated state.

FIG. 2B is a perspective view of the balloon in the inflated state.

### DETAILED DESCRIPTION

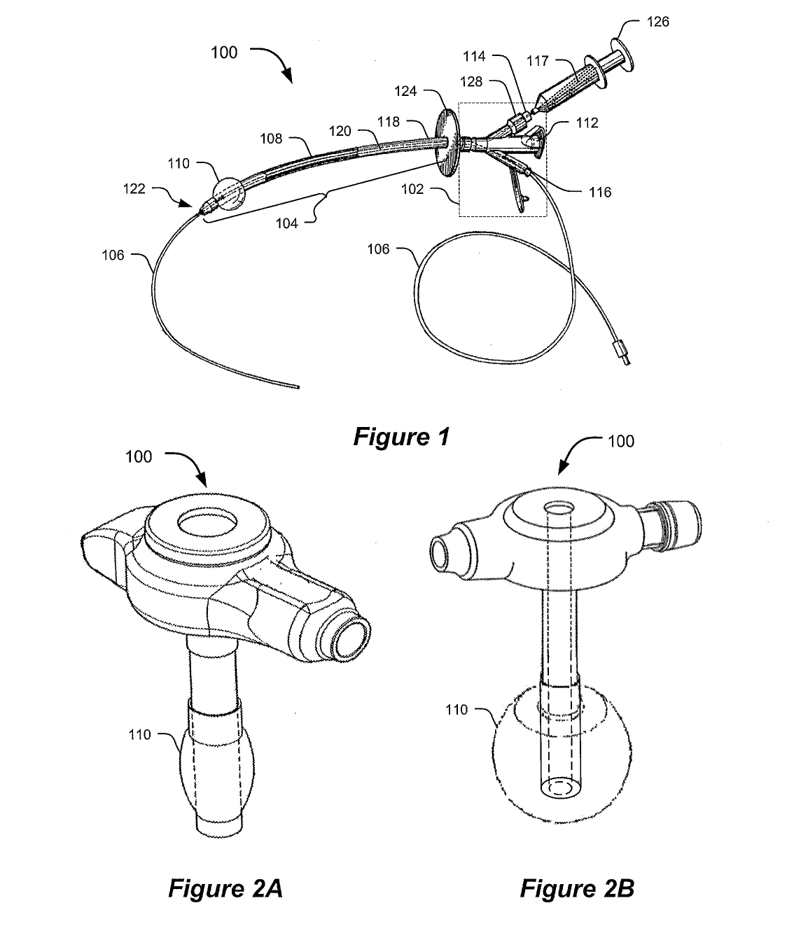

A perspective view of an exemplary enteral feeding device 100 in accordance with the present disclosure is shown in FIG. 1. The enteral feeding device 100 includes a handling unit 102, an inflation system 104 or internal bolster, and a feeding tube 106. The inflation system 104 is configured to position the device 100 into the stomach through a stoma in the body. The inflation system 104 includes a gastric tube 108 and an inflatable balloon 110.

In one embodiment, the gastric tube 108 includes a flexible tubular portion comprising a biocompatible material. The biocompatible material may include, e.g., one or more heat-resistant materials such as acrylonitrile butadiene styrene, EP46HT-2MED, Noryl resin, or other similar material. The gastric tube 108 may have a distal end and a proximal end that extends to meet the handling unit 102. The handling unit 102 may include one or more lumens and/or one or more ports. For example, in one embodiment shown in FIG. 1, the handling unit 102 may include a handle 112, an inflation port 114, and a feeding port 116. The handle 112 may be ergonomically designed to provide a comfortable grip on the enteral feeding device 100 during operation. The inflation port 114 may be configured to allow flow of an inflation medium 117 to the inflation system 104. The feeding port 116 may be configured to allow the passage of a material, e.g., fluid materials such as one or more nutritional supplements or medicines, via the feeding tube 106 to a desired region inside the body of a patient.

The gastric tube 108 may include a double lumen shaft, e.g., including an inflation lumen 118 and a feeding lumen 120, which may be defined separately (i.e., do not connect) within the walls of the tube 108. The inflation lumen 118 may be in fluid communication with the inflation port 114 and an inflatable member 110, e.g., balloon, which may be located adjacent to the distal end of the gastric tube 108. The inflatable balloon 110 may be made of any suitable flexible biocompatible material. In some embodiments, the inflatable balloon comprises medical grade silicone, which may be beneficial due to its unique properties, including low thermal conductivity, low chemical reactivity, thermal stability, and ability to repel water. Other biocompatible materials may be used for the inflatable balloon including, but not limited to, polyurethane and other suitable elastomeric polymers.

Further referring to FIG. 1, the feeding lumen 120 may be in fluid communication with the feeding port 116, and may extend distally through a lumen in the inflatable balloon 110. The feeding lumen 120 forms a distal opening 122 to the balloon 110 at the distal end of the gastric tube 108. The feeding port 116 may receive the feeding tube 106, which may be advanced distally out of the distal opening 122 in the gastric tube 108 via the feeding lumen 120.

While FIG. 1 illustrates one type of device, the present disclosure may be used with any enteral feeding device, e.g., comprising an inflatable member such as a balloon, including commercial devices known in the art.

According to some embodiments of the present disclosure, the gastric tube 108 may be introduced within a stoma in the body of a patient from the distal end of the tube 108, while having the balloon 110 in a deflated state. The balloon 110 may be advanced through the stoma to bear against the wall of the stomach surrounding the stoma. Once the balloon 110 is properly placed, a positive pressure may be applied by introducing inflation medium 117 into the balloon 110 via the inflation lumen 118, causing the balloon 110 to expand. The outer diameter of the expanded balloon 110 is thus greater than the diameter of the stoma, allowing the balloon 110 to remain in place against the wall of the stomach. Such positioning of the balloon 110 within the stoma may seal or substantially seals the stoma against leakage or seeping of gastric juices from the stomach.

FIGS. 2A and 2B show deflated and inflated states, respectively, of a balloon 210 of a device 200 according to another embodiment. While device 200 of FIGS. 2A-2B may have a different structure than the device 100 of FIG. 1, device 200 may include one or more of the features shown in FIG. 1, including, but not limited to, an inflation port, an inflation lumen, an inflation medium, a feeding port, a feeding tube, a feeding lumen, a gastric tube, etc. FIGS. 1 and 2A-2B are not intended to be limiting of embodiments provided by the disclosure herein.

In some embodiments, the device 100 may include a stopper 124 as shown in FIG. 1 to further reduce or prevent leakage of gastric juices. For example, the stopper 124 may bear against the outer surface of the stomach, e.g., forming a seal with the stomach surface. The stopper 124 may be affixed to the gastric tube 108, e.g., permanently or removably affixed, or may also be controllably movable on the gastric tube 108. The stopper 124 may be manipulated, for example, to slide along the exterior of the gastric tube 108.

Subsequently, the feeding tube 106 may be inserted through the feeding port 116 and advanced into a desired region in the body, e.g., the stomach, via the feeding lumen 120, to supply one or more materials, e.g., nutritional fluids, to the patient as needed. After a desirable or required supply of fluent materials is complete, the balloon 110 may be deflated by applying a negative pressure to the inflation lumen 118 to withdraw the inflation medium 117, so that the gastric tube 108 can be removed from the stoma.

Application of positive and negative pressures to the inflation lumen 118 as described above to inflate and deflate the balloon 110 may be performed in a conventional manner, such as by using a syringe 126, or any suitable device capable of controlling and applying the pressure to the inflation lumen 118. The syringe 126 may comprise inflation medium 117 as shown in FIG. 1. The syringe 126 may communicate with the inflation lumen 118 through a valve 128 coupled to the inflation port 114. The valve 128 may control introduction and/or withdrawal of pressure, e.g., inflation medium 117, from the inflation lumen 118. In some embodiments, the valve 128 and the inflation lumen 118 are isolated from the feeding lumen 120 of the gastric tube 108. Separating the inflation lumen 118 from the feeding lumen 120 may avoid or prevent cross-contamination, e.g., of inflation medium 117 and nutritional or medicinal materials delivered to the patient via the feeding lumen 120.

As indicated above, water or saline used as the inflation medium may pass or leech through the walls of a silicone balloon over time, for example, causing the balloon volume to decrease and deflate the balloon. This decrease in water/saline volume may result in the enteral feeding device being unintentionally dislocated or removed from the stomach with potentially adverse consequences for the patient.

In at least some embodiments of the present disclosure, the inflation medium 117 may comprise a fluid other than water or saline, e.g., to reduce or avoid leeching of the inflation medium 117 through the walls of the inflatable balloon 110, and reduce and/or minimize unintentional removal of enteral feeding device 100. The inflation medium may include a biphase material, e.g., a gel, hydrogel, emulsion, or other continuous or discontinuous biphase material.

In some embodiments, the inflation medium 117 comprises a polymer or polymer mixture. As used herein, the term “polymer” includes compounds with at least one repeating structural unit or monomer. Polymers suitable for the present disclosure may include, for example homopolymers, copolymers, block copolymers, cross-linked polymers, cationic polymers, anionic polymers, macromolecules, and/or biopolymers. In some embodiments, the inflation medium may comprise a hydrogel, for example, having a substantially cross-linked structure. Polymers according to the present disclosure may be naturally-derived and/or synthetic, and may be organic and/or inorganic. The polymer(s) used for the inflation medium 117 may be configured to keep the valve 128 free or sufficiently free from clogging during injection of the inflation medium 117 into the inflation port 114. For example, the inflation medium 117 may comprise a polymer or polymer mixture that does not substantially crystallize or otherwise leave a residue as a result of passing through the inflation port 114.

In some embodiments of the present disclosure, the inflation medium 117 may comprise at least one hydrophilic polymer. A hydrophilic polymer generally comprises one or more polar or charged functional groups that render the polymer soluble in water. Examples of hydrophilic polymers suitable for the present disclosure include, but are not limited to, polymers comprising one or more acrylate or amine functional groups such as, e.g., acrylic acid, maleic anhydride polymers, allylamine, ethyleneimine, oxazoline, and other copolymers having similar functional groups in their main or side chains. Other exemplary hydrophilic or water-soluble polymers include, but are not limited to, polyvinyl alcohol (PVA), polyethylene glycol (PEG), dextran, and polyvinylpyrrolidone (PVP). Polymer mixtures, including mixtures of one or more hydrophilic polymers, are further contemplated.

In some embodiments of the present disclosure, the inflation medium 117 comprises one or more thermosensitive polymers, also known as phase transition polymers. Examples of phase transition polymers include, for example, poloxamers and pluronics. Poloxamers generally refer to triblock copolymers having an amphiphilic structure, e.g., comprising hydrophobic and hydrophilic polymer chains. Poloxamers may be generally liquid at low temperatures and increase in viscosity with increasing temperature, e.g., transitioning from a liquid into a gel or solid. Poloxamers may comprise, for example, a repeating structure comprising one hydrophobic chain of polyoxypropylene and two hydrophilic chains of polyoxyethylene. Examples of poloxamers include products by Pluromed such as, e.g., P188 and P407.

For example, a phase transition polymer used as the inflation medium 117 according to the present disclosure may increase in viscosity upon injection into the balloon 110 for inflation when the polymer is warmed by body heat through the material of the balloon 110. Likewise, decreasing temperature may decrease the viscosity of the polymer used as inflation medium 117, e.g., transitioning the inflation medium 117 from a solid or gel into a liquid. In case the polymer is water-soluble, the phase transition polymer may decrease in viscosity upon mixing with cool water. Thus, the aqueous, biocompatible polymer used as inflation medium 117 may be reversible back to a liquid via cooling, for example, by flushing cold water into the balloon 110 from the inflation port 114 via the inflation lumen 118. Any other compatible solvent may be used to cool the polymer to decrease viscosity, e.g., to initiate a phase change. As a result, the gel properties of the poloxamer polymer may be reversed back into a liquid for convenient removal, e.g., via a syringe, to deflate the balloon 110.

The relative change in viscosity may occur gradually, e.g., within several minutes, or may also occur fairly quickly, e.g., within several seconds. Kinetics of the chance in viscosity or phase transition may depend on the composition of the inflation medium 117 and/or temperature(s) to which the inflation medium is exposed. The increase or decrease in polymer viscosity may occur relatively quickly for a given change in temperature, e.g., from room temperature to body temperature.

According to some embodiments, the inflation medium 117 may be configured to adjust according to the inner contour of stomach of the patient, e.g., to ensure that the balloon 110 remains pliable. As a result, discomfort to the patient due to installation of the enteral feeding device 100 may be significantly reduced. Moreover, the inflation medium 117, as discussed above, may limit unintentional and/or unwanted decrease in the balloon 110 volume by leeching or passing through the material comprising the walls of the balloon 110.

The position of the device 100 and/or volume of inflation medium 117 inflating the balloon 110 may be checked periodically, e.g., by the patient, healthcare provider, family member, friend, or other assistant. In some embodiments of the present disclosure, the volume of inflation medium 117 in the balloon 110 may be sufficiently stable to reduce the need to monitor the volume over time, such as in comparison to using water or saline in conventional methods. For example, the volume of inflation medium 117 inflating the balloon 110 may be checked and/or measured twice per week or less frequently, e.g., once per week, or once every two weeks.

Further, in some embodiments, the enteral feeding device 100 may be provided as a kit to the patient or a physician. The kit may include a variety of articles, which are assembled to form the enteral feeding device 100. These articles may include, but not limited to, the feeding tube 106, the syringe 126 pre-filled with the inflation medium 117, and the gastric tube 108, which includes the inflatable member such as balloon 110 and the handling unit 102, each as discussed above. Alternatively or additionally, the kit may include the inflation medium 117 provided in one or more separate containers along with an empty syringe having dimensions same or different from those of the syringe 126. One of skill in the art would understand to include a syringe having appropriate specifications for injecting the inflation medium 117 into the balloon 110 via the inflation lumen 118 in the gastric tube 108.

Although the embodiments described above have been set out in connection with enteral feeding devices, those skilled in the art will understand that the principles set out above can be applied to any enteral medicating device and can be implemented in different ways without departing from the scope of the invention as defined by the claims. In particular, constructional details, including manufacturing techniques and materials, are well within the understanding of those of skill in the art and have not been set out in any detail here. These and other modifications and variations are well within the scope of the present disclosure and can be envisioned and implemented by those of skill in the art.

Moreover, while specific embodiments may have been illustrated and described collectively herein, it should be appreciated that any subsequent arrangement designed to achieve the same or similar purpose may be substituted for the specific embodiments described and shown herein. This disclosure is intended to cover any and all subsequent adaptations or variations of various embodiments. Combinations of the above embodiments, and other embodiments not specifically described herein, will be apparent to those of skill in the art upon reviewing the description.

Other embodiments of the present disclosure will be apparent to those skilled in the art from consideration of the specification and practice of the embodiments disclosed herein. It is intended that the specification and examples be considered as exemplary only, and departure in form and detail may be made without departing from the scope and spirit of the present disclosure as defined by the following claims.

What is claimed is:

1. An enteral feeding system comprising:
a tube having a distal end, a proximal end, and at least one lumen extending therebetween, the tube configured for positioning within a stoma of a human body;
an inflation medium configured to increase in viscosity with increasing temperature;
an inflatable member located adjacent to the distal end of the tube, the inflatable member being configured to pass through the stoma in a deflated state, and to not pass through the stoma in an inflated state; and
an inflation port located proximate to the proximal end of the tube, the inflation port being in fluid communication with the inflatable member via the at least one lumen to enable supply of the inflation medium into the inflatable member, wherein the inflatable member is configured to allow human body heat to warm the inflation medium to be provided therein to increase the viscosity of the inflation medium.

2. The system of claim 1, wherein the inflation medium comprises a liquid capable of transitioning into a gel upon being heated.

3. The system of claim 1, wherein the inflation medium comprises at least one polymer.

4. The system of claim 3, wherein the at least one polymer is naturally-derived or synthetic.

5. The system of claim 3, wherein the inflation medium comprises at least one homopolymer, copolymer, block copolymer, cross-linked polymer, or hydrophilic polymer.

6. The system of claim 1, wherein the inflation medium comprises at least one of acrylic acid, a maleic anhydride polymer, allylamine, ethyleneimine, oxazoline, polyvinyl alcohol, polyethylene glycol, dextran, or polyvinylpyrrolidone.

7. The system of claim 3, wherein the inflation medium comprises at least one thermosensitive polymer.

8. An enteral feeding system comprising:
a gastric tube having a distal end, a proximal end, and tube walls defining an inflation lumen and a feeding lumen, the feeding lumen opening at the distal end of the tube, wherein the gastric tube includes:
an inflatable member located adjacent to the distal end of the tube; and
an inflation port and a feeding port, each located adjacent to the proximal end of the tube, wherein the inflation port is in fluid communication with the inflatable member through the inflation lumen, and wherein the feeding port is in fluid communication with the feeding lumen;
a syringe configured to house a volume of a fluid, wherein the fluid comprises at least one thermosensitive polymer configured to increase in viscosity with increasing temperature; and
a feeding tube configured to attach in fluid communication with the feeding port, the feeding tube being capable of extending into a stomach through the feeding lumen.

9. The system of claim 8, wherein the fluid is configured to increase in viscosity into a gel or a solid upon being heated.

10. The system of claim 8, wherein the fluid is water soluble.

11. The system of claim 8, wherein the fluid comprises at least one homopolymer, copolymer, block copolymer, cross-linked polymer, or hydrophilic polymer.

12. The system of claim 8, wherein the at least one thermosensitive polymer is naturally-derived or synthetic.

13. The system of claim 8, wherein the fluid comprises at least one of acrylic acid, a maleic anhydride polymer, allylamine, ethyleneimine, oxazoline, polyvinyl alcohol, polyethylene glycol, dextran, or polyvinylpyrrolidone.

14. The system of claim 8, wherein the fluid comprises at least one poloxamer.

15. The system of claim 8, further comprising at least one container including the fluid.

16. A method of supplying a material to a human body via a stoma, the method comprising:
passing a medical device through the stoma, the medical device comprising:
a tube having a distal end, a proximal end, and first and second lumens extending therebetween;
a fluid including at least one thermosensitive polymer configured to increase in viscosity with increasing temperature;
an inflatable member located adjacent to the distal end of the tube, the inflatable member being configured to pass through the stoma in a deflated state, and to not pass through the stoma in an inflated state; and
an inflation port located proximate to the proximal end of the tube, the inflation port being in fluid communication with the inflatable member via the first lumen;
injecting the fluid into the inflatable member to expand the inflatable member, wherein human body heat causes the fluid to increase in viscosity to secure the medical device at the stoma; and
feeding the fluent materials to the stomach through the second lumen.

17. The method of claim 16, further comprising:
injecting water into the inflatable member to dissolve the fluid in the water; and
deflating the inflatable member by drawing the water and fluid out of the inflatable member through the first lumen.

18. The method of claim 16, wherein the fluid solidifies into a gel upon being heated.

19. The method of claim 16, wherein the fluid comprises at least one homopolymer, copolymer, block copolymer, cross-linked polymer, or hydrophilic polymer.

20. The method of claim 16, wherein the fluid comprises at least one poloxamer.
